(19) United States
(12) Patent Application Publication
(10) Pub. No.: US 2018/0244592 A1
(43) Pub. Date: Aug. 30, 2018

(54) DEHYDROGENATION OF ETHYLBENZENE TO STYRENE

(71) Applicant: HALDOR TOPSØE A/S, Kgs. Lyngby (DK)

(72) Inventors: Poul Erik HØJLUND NIELSEN, Fredensborg (DK); Rasmus Munksgård NIELSEN, Måløv (DK); John Bøgild HANSEN, Humlebæk (DK); Burcin TEMEL MCKENNA, Hellerup (DK)

(73) Assignee: HALDOR TOPSØE A/S, Kgs. Lyngby (DK)

(21) Appl. No.: 15/769,395

(22) PCT Filed: Oct. 24, 2016
(86) PCT No.: PCT/EP2016/075491

§ 371 Date: Apr. 19, 2018

(30) Foreign Application Priority Data
Oct. 28, 2015 (DK) PA 2015 00665

(51) Int. Cl. C07C 5/333; B01J 8/02; B01J 23/745; B01J 23/83; B01J 23/75; B01J 23/755; B01J 23/881; B01J 23/78; B01J 35/00

(52) U.S. Cl. CPC C07C 5/3332; B01J 35/0006; B01J 23/745; B01J 23/83; B01J 23/75; B01J 23/755; B01J 23/881; B01J 23/78; B01J 35/0033; C07C 2523/745; C07C 2523/75; C07C 2523/755; C07C 2523/83; C07C 2523/78; C07C 2523/881; B01J 2208/00442; B01J 8/0285

(57) ABSTRACT

A reactor system for dehydrogenation of ethylbenzene to styrene in a given temperature range T upon bringing a reactant stream including ethylbenzene into contact with a catalytic mixture. The reactor system includes a reactor unit arranged to accommodate the catalytic mixture, the catalytic mixture including catalyst particles in intimate contact with a ferromagnetic material, where the catalyst particles are arranged to catalyze the dehydrogenation of ethylbenzene to styrene. The reactor system moreover includes an induction coil arranged to be powered by a power source supplying alternating current and being positioned so as to generate an alternating magnetic field within the reactor unit upon energization by the power source, whereby the catalytic mixture is heated to a temperature within the temperature range T by means of the alternating magnetic field. Also, a catalytic mixture and a method of dehydrogenating ethylbenzene to styrene.

The present invention relates to a process for the dehydrogenation of ethylbenzene to styrene More particularly, the process comprises heating a catalytic mixture within a reactor unit inductively. Moreover, the present invention comprises a reactor system for dehydrogenation of ethylbenzene to styrene, and a catalytic mixture for catalyzing dehydrogenation of ethylbenzene to styrene.

Current state of the art in industrial processes for dehydrogenation of ethylbenzene are high temperature processes, typically above 600° C., and in all cases also low pressure processes. This is due to the fact that the dehydrogenation reaction C₆H₅CH₂CH₃⇄C₆H₅CHCH₂+H₂ is endothermic requiring about 125 kJ/mole and involves forming more product moles than reactants. Thus, both a temperature increase and low pressure lead to higher conversion. The dehydrogenation of ethylbenzene is normally carried out with steam as diluent, or without diluents. For dehydrogenation of ethylbenzene for production of styrene, current state of the art industrial processes are conducted by using a promoted iron oxide catalyst and with water/steam as carrier gas (diluent) and carbon suppressor.

One of the parasitic reactions in dehydrogenation is carbon formation, which leads to deactivation of the catalyst. Thus, recurrent regenerations of the catalyst may be necessary in certain applications. Carbon formation is not only a problem for the catalyst. Also the material used for the dehydrogenation reactor and for the piping has to be carefully selected, typically by using highly expensive alloys in order to avoid carbon attack resulting in the catastrophic form of corrosion known as metal dusting.

Steam is used in the dehydrogenation process as a heat carrier. Steam needs to be produced by an energy intensive evaporation and has to be condensed out again in the process. The heat of evaporation cannot be fully recuperated. In current processes for conversion of ethylbenzene to styrene, a large amount of steam is added to the ethylbenzene in order to suppress carbon formation on the catalyst, dilute the gas in order to lowering the ethylbenzene partial pressure to favor the equilibrium and finally provide the heat of reaction by acting as a heat carrier.

It is therefore an object of the present invention to provide a process, reactor system and catalytic mixture for dehydrogenation of ethylbenzene to styrene, where maintaining a high stability of the catalytic mixture is achieved.

It is another object of the present invention to provide a process, reactor system and catalytic mixture for dehydrogenation of ethylbenzene to styrene, which are simple and energy efficient and which at the same time enables maintaining high stability of the catalyst.

It is also an object of the present invention to increase the energy efficiency of the dehydrogenation of ethylbenzene to styrene.

It is also an object of the present invention to provide a process, reactor system and catalytic mixture wherein the reaction temperature is controlled accurately. Preferably, the temperature of the process is lowered compared to hitherto known reactions; hereby, the thermodynamic potential for dehydrogenation is increased and parasitic reactions, such as coking of the catalytic mixture and/or cracking of the reactant stream are reduced.

The present invention solves one or more of the above mentioned problems.

An aspect of the present invention relates to a reactor system for dehydrogenation of ethylbenzene to styrene in a given temperature range T upon bringing a reactant stream comprising ethylbenzene into contact with a catalytic mixture. The reactor system comprises a reactor unit arranged to accommodate the catalytic mixture, where the catalytic mixture comprising catalyst particles in intimate contact with a ferromagnetic material, where the catalyst particles are arranged to catalyze the dehydrogenation of ethylbenzene to styrene. The reactor system moreover comprises an induction coil arranged to be powered by a power source supplying alternating current and being positioned so as to generate an alternating magnetic field within the reactor unit upon energization by the power source, whereby the catalytic mixture is heated to a temperature within the temperature range T by means of the alternating magnetic field.

In the catalytic mixture, the catalyst particles and ferromagnetic material are in intimate contact. The effect of the catalyst particles and the ferromagnetic material of the system being in intimate contact is that the heat generated in the ferromagnetic material is conducted to the catalytic particles, either directly or indirectly via a reactant stream during operation, over a short distance. Therefore, the heating within the reactor system takes place at or very close to the catalyst particles. Thus, the temperature of the reactant stream may be lower, when the reactant stream reaches the catalytic mixture than when it leaves the catalytic mixture within the reactor system. This gives less problems with cracking and coking within the reactor system.

The term “catalyst particles in intimate contact with a ferromagnetic material” is meant to denote that catalyst particles are in substantial proximity to the ferromagnetic material, such as in a physical mixture together with the ferromagnetic material, in physical contact with the ferromagnetic material or supported on the ferromagnetic material, possibly via an oxide.

A key element which the present invention addresses is the issue of supplying the heat needed to carry out the dehydrogenation reaction. In a conventional process of dehydrogenating ethylbenzene to styrene, this is done by using steam as a carrier gas. The reaction is often carried out in two or three adiabatic catalytic beds with reheating to a temperature of 600-650° C. between the beds. Steam is used as a carrier gas, as a diluent and carbon suppressor. By the reactor system of the invention it is rendered possible to dehydrogenate ethylbenzene to styrene with a considerably reduced steam consumption, in that steam is no longer needed as a heat carrier. Instead, the heat for the endothermic dehydrogenation reaction is provided by induction heating. The Curie temperature of the ferromagnetic material may be close to, above or far above the upper limit of the given temperature range T. However, the Curie temperature could be slightly lower than the upper limit of the given temperature range T, in that the reactant gas stream entering the reactor system may be heated to a temperature above the Curie temperature before entering the reactor system, thereby providing an upper limit of the temperature range T—in an upstream part of the reactor unit—which is higher than that obtainable by induction heating. As used herein, the term “temperature range T” is meant to denote a desired range of temperatures, typically up to an upper limit thereof, at which the dehydrogenation reaction is to take place within the reactor system during operation.

Preferably, the coercivity of the ferromagnetic material is high, so that the amount of heat generated within the ferromagnetic material and dissipated by the external field in reversing the magnetization in each magnetization cycle is high.

As used herein, a material of “high magnetic coercivity”, _{B}H_{C}, is seen as a “hard magnetic material” having a coercivity _{B}H_{C} at or above about 20 kA/m, whilst a material of “low magnetic coercivity” is seen as a “soft magnetic material” having a coercivity _{B}H_{C} at or below about 5 kA/m. It should be understood that the terms “hard” and “soft” magnetic materials are meant to refer to the magnetic properties of the materials, not their mechanical properties.

It should be noted that steam in some amount is still essential in the process for suppressing carbon formation on the catalyst. However, by using the reactor system of the invention the steam to carbon ratio may be reduced considerably compared to a reactor system using steam as heat carrier.

Ferromagnetic material provides for further advantages, such as:
A ferromagnetic material absorbs a high proportion of the magnetic field, thereby making the need for shielding less or even superfluous.
Heating of ferromagnetic materials is relatively faster and cheaper than heating of non-ferromagnetic materials. A ferromagnetic material has an inherent or intrinsic maximum temperature of heating, viz. the Curie temperature.

Therefore, the use of a catalyst mixture which is ferromagnetic ensures that an endothermic chemical reaction is not heated above a specific temperature, viz. the Curie temperature.

Another advantage of the invention is that the temperature of the reactor unit can be kept lower than the temperature of the conventionally used adiabatic reactor. The lower temperature is beneficial for the overall yield and selectivity of the process and required regenerations for carbon removal will be less frequent since parasitic reactions like coking and cracking are reduced. The amount of steam may be reduced since its primary function as heat carrier no longer exists. The reduced amount of steam used in the process further improves the economics of the process, and problems with potassium transport present in the hitherto known processes are reduced. Further advantages comprise the possibility of tuning the exit temperature, which increase the thermodynamic potential for dehydrogenation.

The induction coil may e.g. be placed within the reactor unit or around the reactor unit. If the induction coil is placed within the reactor unit, it is preferable that it is positioned at least substantially adjacent to the inner wall(s) of the reactor unit in order to surround as much of the catalytic mixture as possible. In the cases, where the induction coil is placed within the reactor unit, windings of the reactor unit may be in physical contact with catalyst mixture. In this case, in addition to the induction heating, the catalyst mixture may be heated directly by ohmic/resistive heating due to the passage of electric current through the windings of the induction coil. The reactor unit is typically made of non-ferromagnetic material.

In conclusion, the invention provides a reactor system arranged to carry out dehydrogenation of ethylbenzene to styrene cheaper and with better selectivity than current reactor systems. Moreover, the lifetime of the catalyst will be improved due to the lower average operation temperature within the reactor system.

In an embodiment, the given temperature range T is the range between about 450° C. and about 650° C. or a sub-range thereof. Preferably, the Curie temperature of the ferromagnetic material is above 450° C., e.g. between 450° C. and 650° C. In an embodiment, the Curie temperature of the ferromagnetic material equals an operating temperature at substantially the upper limit of the given temperature range T of the dehydrogenation reaction. Hereby, it is ensured that the dehydrogenation reaction is not heated above a specific temperature, viz. the Curie temperature. Thus, it is ensured that the dehydrogenation reaction will not run out of control.

In an embodiment, the induction coil is placed within the reactor unit or around the reactor unit. The coil may e.g. be made of kanthal.

In an embodiment, the catalyst particles are supported on the ferromagnetic material.

In an embodiment, the ferromagnetic material comprises one or more ferromagnetic macroscopic supports susceptible for induction heating, where the one or more ferromagnetic macroscopic supports is/are coated with an oxide and where the oxide is impregnated with catalyst particles.

The oxide may also be impregnated with ferromagnetic particles. Thus, when the catalyst particles are subjected to a varying magnetic field, both the ferromagnetic macroscopic support and the ferromagnetic particles impregnated into the oxide of the ferromagnetic macroscopic support are heated. Whilst the ferromagnetic macroscopic support heats the catalyst particles from within, the ferromagnetic particles heats from the outside of the oxide. Thereby, a higher temperature and/or a higher heating rate are/is achievable.

As used herein, the term “macroscopic support” is meant to denote a macroscopic support material in any appropriate form providing a high surface area. Non-limiting examples are metallic elements, monoliths or miniliths. The macroscopic support may have a number of channels; in this case it may be straight-channeled or a cross-corrugated element. The material of the macroscopic support may be porous or the macroscopic support may be a solid. The word “macroscopic” in “macroscopic support” is meant to specify that the support is large enough to be visible to the naked eye, without magnifying devices.

In an embodiment, the catalyst particles and ferromagnetic particles powder are mixed and treated to provide bodies of catalytic mixture. The magnitude of particles is in the micrometer scale, such that a characteristic size of catalyst and/or ferromagnetic particles is larger than 0.1 μm. The size of particles of ferromagnetic material powder needs to be sufficient for ferromagnetic heating to take place. This is e.g. described in “Magnetic multi-granule nanoclusters: A model system that exhibits universal size effect of magnetic coercivity”, by Ji Sung Lee et al, Scientific Report, published 17 July 2015 (see e.g. FIG. 1). Preferably, the smallest outside dimension of the bodies is between about 2-3 mm and about 8 mm. The ratio between catalyst particles and ferromagnetic particles may e.g. be 1:1. Alternatively, the catalytic mixture may comprise more ferromagnetic particles than catalyst particles, depending on the bodies and the reaction conditions.

In an embodiment, the catalytic mixture comprises bodies of catalyst particles mixed with bodies of ferromagnetic material, wherein the smallest outside dimension of the bodies is in the order of about 1-2 mm or larger. Preferably, the smallest outside dimension of the bodies is between about 2-3 mm and about 8 mm. The bodies of catalyst particles are e.g. extrudates or miniliths. The bodies of ferromagnetic material may e.g. be iron spheres.

In an embodiment, the catalytic mixture has a predetermined ratio between the catalyst particles and the ferromagnetic material. In an embodiment, the predetermined ratio between the catalyst particles and the ferromagnetic material is a predetermined graded ratio varying along a flow direction of the reactor. Hereby, it is possible to control the temperature in different zones of the reactor. A radial flow reactor may be used; in this case, the predetermined ratio varies along the radial direction of the reactor. Alternatively, an axial flow reactor may be used. When the catalytic mixture has a graded ratio, it is possible to operate the reactor system so that the temperature is increased along the flow direction, viz. over the bed length, hereby increasing the thermodynamic potential for dehydrogenating ethylbenzene. In this manner, problems with potassium transport present in the hitherto known processes are reduced.

Another aspect of the invention relates to a catalytic mixture arranged for catalyzing dehydrogenation of ethylbenzene in a reactor in a given temperature range T upon bringing a reactant stream comprising ethylbenzene into contact with the catalytic mixture, the catalytic mixture comprising a catalyst particles in intimate contact with a ferromagnetic material, where the catalyst particles are arranged to catalyze the dehydrogenation of ethylbenzene to styrene. The catalytic mixture may have a predetermined ratio between the catalyst particles and the ferromagnetic material.

In an embodiment, the Curie temperature of the ferromagnetic material substantially equals an operating temperature at substantially the upper limit of the given temperature range T of the dehydrogenation reaction. Alternatively, the Curie temperature could be slightly lower than the upper limit of the given temperature range T, in that the reactant gas stream entering the reactor system may be heated to a temperature above the Curie temperature before entering the reactor system, thereby providing an upper limit of the temperature range T—in an upstream part of the reactor unit—which is higher than that obtainable by induction heating.

In an embodiment, the ferromagnetic material is an alloy comprising iron, an alloy comprising iron and chromium, an alloy comprising iron, chromium and aluminum, an alloy comprising iron and cobalt, an alloy comprising iron, aluminum, nickel and cobalt, or magnetite. In an embodiment, the catalyst particles comprise a carrier impregnated with an iron oxide and/or a potassium oxide, optionally promoted by a cerium oxide and/or a molybdenum oxide.

In an embodiment, the catalyst particles are is supported on the ferromagnetic material. For example, the ferromagnetic material comprises one or more ferromagnetic macroscopic supports susceptible for induction heating, where the one or more ferromagnetic macroscopic supports is/are coated with an oxide and where the oxide is impregnated with catalyst particles. Non-limiting examples of ferromagnetic macroscopic supports coated with an oxide, which in turn is impregnated with catalyst particles, are metallic elements, monoliths or miniliths.

In an embodiment, catalyst particles and ferromagnetic particles are mixed and treated to provide bodies of catalytic mixture, the bodies having a predetermined ratio between catalyst particles and ferromagnetic material. In an embodiment, the catalytic mixture comprises bodies of catalyst particles mixed with bodies of ferromagnetic material. Such bodies may e.g. be pellets, extrudates or miniliths.

In an embodiment, the catalytic mixture has a predetermined ratio between the catalyst particles and the ferromagnetic material. The predetermined ratio between the catalyst particles and the ferromagnetic material may be a predetermined graded ratio varying along a flow direction of the reactor. Hereby, it is possible to control the temperature in different zones of the reactor. A radial flow reactor may be used; in this case, the predetermined ratio varies along the radial direction of the reactor. Alternatively, an axial flow reactor may be used.

Another aspect of the invention relates to a method for dehydrogenating of ethylbenzene in a given temperature range T in a reactor system, where the reactor system comprises a reactor unit arranged to accommodate a catalytic mixture, and where the catalytic mixture comprising catalyst particles in intimate contact with a ferromagnetic material. The catalyst particles are arranged to catalyze the dehydrogenation of ethylbenzene to styrene. An induction coil is arranged to be powered by a power source supplying alternating current and positioned so as to generate an alternating magnetic field within the reactor unit upon energization by the power source, whereby the catalytic mixture is heated to a temperature within the given temperature range T by means of the alternating magnetic field. The method comprises the steps of:

Generating an alternating magnetic field within the reactor unit upon energization by a power source supplying alternating current, the alternating magnetic field passing through the reactor unit, thereby heating catalytic mixture by induction of a magnetic flux in the material;
(ii) bringing a reactant stream comprising ethylbenzene into contact with the catalyst particles;
(iii) heating the reactant stream within the reactor by the generated alternating magnetic field; and
(iv) letting the reactant stream react in order to provide a styrene product stream to be outlet from the reactor.

The sequence of the steps (i) to (iv) is not meant to be limiting. Steps (ii) and (iii) may happen simultaneously, or step (iii) may be initiated before step (ii) and/or take place at the same time as step (iv). Advantages as explained in relation to the reactor system and the catalytic mixture also apply to the method for dehydrogenating ethylbenzene to styrene. The catalytic mixture may have a predetermined ratio between the catalyst particles and the ferromagnetic material. In an embodiment, the temperature range T is the range from between about 450° C. and about 650° C. or a subrange thereof. In an embodiment, the reactant stream is preheated in a heat exchanger prior to step (ii).

The Curie temperature of the ferromagnetic material may be equal to or above an upper limit of the given temperature range T of the dehydrogenation reaction. Alternatively, the Curie temperature could be slightly lower than the upper limit of the given temperature range T, in that the reactant gas stream entering the reactor system may be heated to a temperature above the Curie temperature before entering the reactor system, thereby providing an upper limit of the temperature range T—in an upstream part of the reactor unit—which is higher than that obtainable by induction heating.

In an embodiment, the mass ratio between steam and ethylbenzene in the reactant stream is a ratio of steam/ethylbenzene between 0.5 and 1.5 (wt/wt). Thus, the method of the invention provides for a reduced usage of steam and thus lowers the operating expenses compared to a corresponding dehydrogenation process wherein heat is supplied to the process by use of steam only.

### BRIEF DESCRIPTION OF THE FIGURES

The invention will now be explained in more detail by reference to the accompanying figures, wherein:

FIG. 1 shows temperature profiles of a reactor unit heated by convective/conductive and/or radiation heating, and induction heating, respectively;

FIG. 2 shows temperature profiles along the length of an inductively heated axial reactor unit according to the invention; and

FIGS. 3a and 3b show schematic drawings of two embodiments of a reactor system;

### DETAILED DESCRIPTION OF THE FIGURES

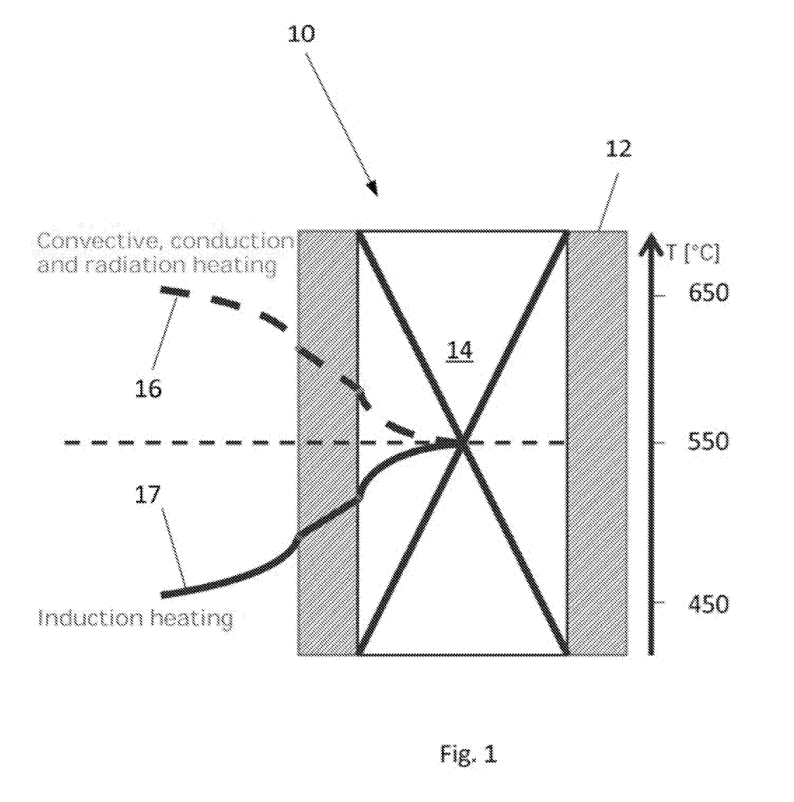

FIG. 1 is a graph showing temperature profiles of a reactor unit 10 heated by convective/conductive and/or radiation heating, and induction heating, respectively, during an endothermic reaction within the reactor unit 10. The temperature profiles in FIG. 1 are indicated together with a schematic cross-section through a reactor unit 10 having walls 12 holding a catalyst bed 14 with a catalyst mixture for endothermic reactions. In the case of induction heating, the catalyst mixture in the catalyst bed 14 is susceptible to inductive heating. Means for heating the reactor unit 10 and/or the catalyst bed 14 are not shown. In the case of convective, conduction and/or radiation heating, the means for heating could e.g. be fired burners; means for induction heating would typically be an electromagnet, e.g. an induction coil. A temperature scale is indicated at the right side of FIG. 1. The reactor unit 10 is an axial flow reactor unit and the temperature profiles shown in FIG. 1 indicate the temperatures at the center of the catalyst bed within the reactor unit. The horizontal dotted line indicates a temperature of 550° C. at the centre of the catalyst bed both in the case of convective, conduction and radiation heating (curve 16) and induction heating (curve 17).

The dotted curve 16 indicates the temperatures outside the reactor unit, at the reactor unit walls as wells as within the catalyst bed 14 when heated by convective/conductive and/or radiation heating, whilst the solid curve 17 indicate the temperatures outside the reactor unit, at the reactor unit walls as well as within the catalyst bed 14 when heated by convective/conductive and/or radiation heating, and induction heating, respectively.

It is clear from FIG. 1, that in the case of convective/conductive and/or radiation heating, the temperature is higher outside the wall 12 than within the wall 12, and that the temperature within the catalyst bed 14 is lower than that at the wall 12. At the center of the catalyst bed, the temperature is at its lowest. This is because the temperature at the heat source must be higher than the reaction zone and due to the temperature loss through the walls and due to the endothermic nature of the reaction within the reactor unit 10. In contrast, the temperature profile as indicated by the curve 17 shows that for induction heating the temperature is higher at the wall 12 compared to outside the reactor unit, whilst the temperature inside the catalyst bed increases from the wall 12 to the center of the catalyst bed 14.

In general, performing endothermic reactions is limited by how efficiently heat can be transferred to the reactive zone of the catalyst bed 14. Conventional heat transfer by convection/conduction/radiation can be slow and will often meet large resistance in many configurations. Moreover, heat losses within the walls of the reactor play a role.

In contrast, when heat is deposited inside the catalyst bed 14 by the induction concept, the catalyst bed will be the hottest part of the reactor 10 in contrast to conventional heating where the exterior heat source has to be significantly hotter than the internal part to have a driving mechanism for the heat transfer.

The reactant stream comprising ethylbenzene and entering the reactor unit has typically been preheated by passing through a feed effluent heat exchanger and arrives into the catalyst bed at a temperature of about 500° C. The flow direction in a radial flow reactor could be from the outside to the centre of the reactor unit or vice versa. The streams are heated to a temperature above 600° C. and during this process, the catalytic conversion of ethylbenzene to styrene takes place. Assuming a H₂O:ethylbenzene ratio of 4, an overall pressure of 1 bara and conversion into equilibrium at 600° C., then 60% of the ethylbenzene will be converted to styrene. Conversion of 1 ton of ethylbenzene to styrene requires 328 kWh and the heating of the reaction mixture will require about 200 kWh. An efficient feed effluent heat exchanger will reduce this amount considerably. The converted gas stream exiting the reactor unit 110 may pass through a feed effluent heat exchanger and may be further cooled.

The process as described may also apply for dehydrogenation of amylenes to isoprene.

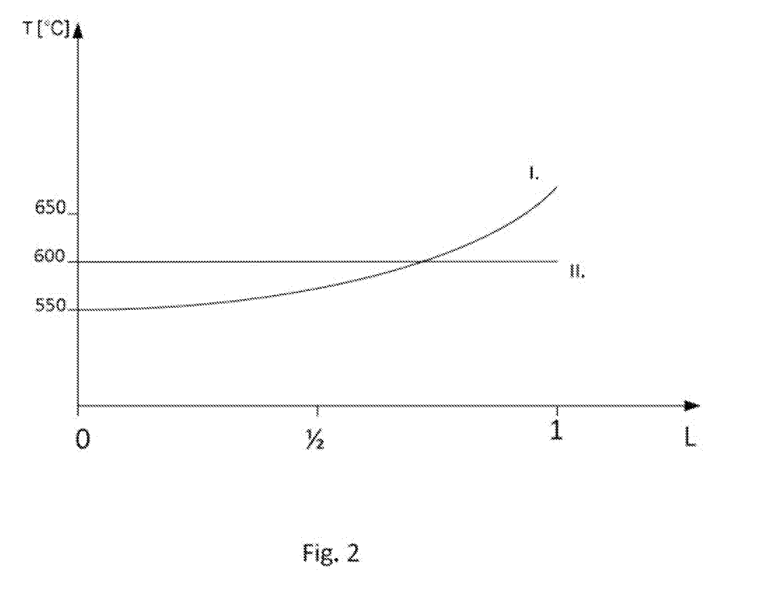

FIG. 2 shows temperature profiles along the length of an inductively heated axial reactor unit according to the invention. FIG. 2 shows two different temperature profiles: an isothermal profile I. and an increasing temperature profile II, along the axial direction of the reactor unit. The reactant stream reaches the catalytic mixture at the reactor length L=0 and leaves the catalytic mixture at the reactor length L=1. In the isothermal profile I. the temperature is held constant throughout the reactor length. This is achievable by designing the induction coil and/or the catalytic mixture accordingly. In the temperature profile II., the temperature increases along the path of the reactant stream through the reactor unit. This is advantageous, in that a relatively low inlet temperature (at L=0), reduces the risk of cracking of the reactant stream, and in that a high temperature towards the end of the reactor unit (L=1) provides an improved thermodynamic equilibrium for the dehydrogenation reaction. In the temperature profile II., it is noted that the maximum reactant stream temperature is the outlet temperature. Even though FIG. 2 is shown for an axial flow reactor unit, similar profiles are relevant for radial flow reactor units along the path of the reactant stream through the catalytic mixture.

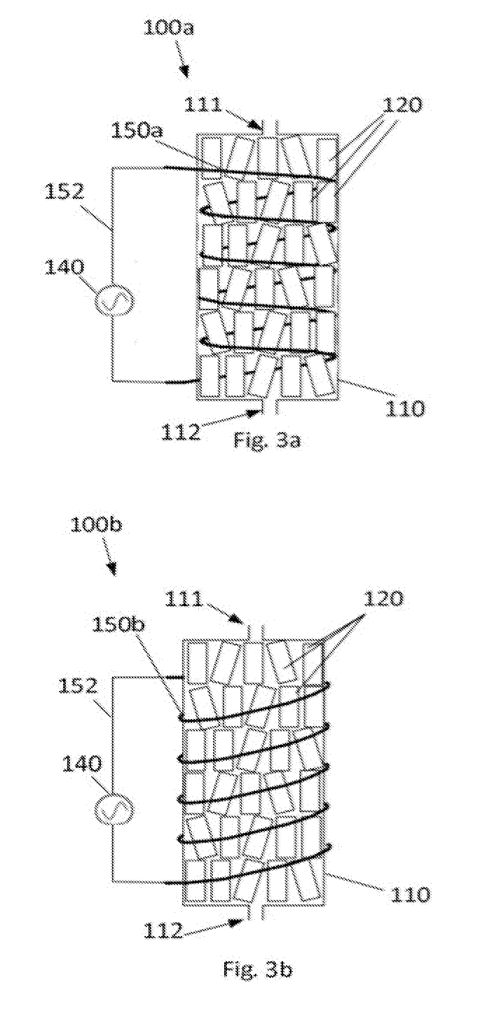

FIGS. 3a and 3b show schematic drawings of five embodiments 100a and 100b, of a reactor system. In FIGS. 3a and 3b, similar features are denoted using similar reference numbers.

FIG. 3a shows an embodiment of the reactor system 100a for carrying out dehydrogenation of ethylbenzene to styrene upon bringing a reactant stream comprising ethylbenzene into contact with a catalytic mixture 120. The reactor system 100a comprises a reactor unit 110 arranged to accommodate a catalytic mixture 120 comprising catalyst particles in intimate contact with a ferromagnetic material, where the catalyst particles are arranged to catalyze the dehydrogenation of ethylbenzene to styrene and the ferromagnetic material is ferromagnetic at least at temperatures up to about 500° C. or 700° C.

Reactant is introduced into the reactor unit 110 via an inlet 111, and reaction products formed on the surface of the catalytic mixture 120 are outlet via an outlet 112.

The reactor system 100a further comprises an induction coil 150a arranged to be powered by a power source 140 supplying alternating current. The induction coil 150a is connected to the power source 140 by conductors 152. The induction coil 150a is positioned so as to generate an alternating magnetic field within the reactor unit 110 upon energization by the power source 140. Hereby the catalytic mixture 120 is heated to a temperature within a given temperature range T relevant for dehydrogenation of ethylbenzene, such as between 450° C. and about 650° C., by means of the alternating magnetic field.

The induction coil 150a of FIG. 3a is placed substantially adjacent to the inner surface of the reactor unit 110 and in physical contact with the catalytic mixture 120. In this case, in addition to the induction heating provided by the magnetic field, the catalyst particles 120 adjacent the induction coil 150a are additionally heated directly by ohmic/resistive heating due to the passage of electric current through the windings of the induction coil 150a. The induction coil 150a may be placed either inside or outside the catalyst basket (not shown) supporting the catalytic mixture 120 within the reactor unit 110. The induction coil is preferably made of kanthal.

The catalytic mixture 120 may be divided into sections (not shown in the figures), where the ratio between the catalytic material and the ferromagnetic material is varies from one section to another. At the inlet of the reactor unit 110, the reaction rate is high and the heat demand is large; this may be compensated for by having a relatively large proportion of ferromagnetic material compared to the catalytic material. The ferromagnetic material may also be designed to limit the temperature by choosing a ferromagnetic material with a Curie temperature close to the desired reaction temperature.

Placing the induction coil 150a within the reactor unit 110 ensures that the heat produced due to ohmic resistance heating of the induction coil 150a remains useful for the dehydrogenation reaction. However, having an oscillating magnetic field within the reactor may cause problems, if the materials of the reactor unit 110 are magnetic with a high coercivity, in that undesirably high temperatures may be the result. This problem can be circumvented by cladding the inside of the reactor unit 110 with materials capable of reflecting the oscillating magnetic field. Such materials could e.g. be good electrical conductors, such as copper. Alternatively, the material of the reactor unit 110 could be chosen as a material with a very low coercivity. Alternatively, the induction coil 150 could be wound as a torus.

To make the catalyst bed susceptible for induction, different approaches may be applied. One approach is to support the catalyst particles on the ferromagnetic material. For example, the ferromagnetic material comprises one or more ferromagnetic macroscopic supports susceptible for induction heating. The one or more ferromagnetic macroscopic supports is/are coated with an oxide and the oxide is impregnated with catalyst particles. Another approach is to mix catalyst particles and ferromagnetic particles and treat the mixture to provide bodies of catalytic mixture. Additionally or alternatively, the catalytic mixture comprises bodies of catalyst particles mixed with bodies of ferromagnetic material, wherein the smallest outside dimension of the bodies are in the order of about 1-2 mm or larger.

The styrene catalyst particles may be based upon iron oxides and potassium oxides along with (an)other metal oxide(s), such as cerium oxide and/or a molybdenum oxide, as promoter(s). The catalyst particles may have a certain content of magnetite, Fe₃O₄, which is ferromagnetic with a Curie temperature of 581° C. This is a typical temperature within a styrene reactor. It is possible to heat the magnetite crystals up to a temperature of 581° C. In a case, where this is not sufficient for obtaining a reasonable conversion, the catalyst particles may be mixed with a ferromagnetic material with a high coercivity and a high Curie temperature, such as AlNiCo or Permendur.

The catalytic mixture preferably has a predetermined ratio between the catalyst particles and the ferromagnetic material. This predetermined ratio may be a graded ratio varying along a flow direction of the reactor.

In another approach, ferromagnetic macroscopic supports are coated with an oxide impregnated with the catalytically active particles. This approach offers a large versatility compared to the ferromagnetic nanoparticles in the catalyst, as the choice of catalytic active phase is not required to be ferromagnetic.

In addition to the possibility of delivering heat directly to the catalyst particles, induction heating offers a fast heating mechanism, which potentially could make upstart of a dehydrogenation reactor relative fast.

FIG. 3b shows another embodiment 100b of the reactor system for carrying out dehydrogenation of ethylbenzene to styrene upon bringing a reactant stream comprising ethylbenzene into contact with a catalytic mixture 120. The reactor unit 110 and its inlet and outlet 111, 112, the catalytic mixture 120, the power source 140 and its connecting conductors 152 are similar to those of the embodiment shown in FIG. 3a.

In the embodiment of FIG. 3b, an induction coil 150b is wound or positioned around the outside of the reactor unit 110.

In both embodiments shown in FIGS. 3a-3b, the catalytic mixture can be any catalytic mixture according to the invention. Thus, the catalytic mixture may be in the form of catalyst particles supported on the ferromagnetic material, e.g. where in the form of ferromagnetic macroscopic support(s) coated with an oxide, where the oxide is impregnated with catalyst particles, miniliths, a monolith, or bodies produced from a mixture of catalyst particles and ferromagnetic particles. Thus, the catalytic mixture is not limited to a catalytic mixture having relative size as compared to the reactor system as shown in the figures. Moreover, when the catalytic mixture comprises a plurality of macroscopic supports, the catalytic mixture would typically be packed so as to leave less space between the macroscopic supports than shown in the FIGS. 3a and 3b. Furthermore, in the two embodiments shown in FIGS. 3a and 3b, the reactor unit 110 is made of non-ferromagnetic material. In the two embodiments shown in FIGS. 3a and 3b, the power source 140 is an electronic oscillator arranged to pass a high-frequency alternating current (AC) through the coil surrounding at least part of the catalytic mixture within the reactor system.

Even though FIGS. 3a and 3b shows an axial or longitudinal flow reactor, the reactor may be a radial flow reactor.

Even though the present invention has been described in connection with dehydrogenation of alkanes, primarily the dehydrogenation of alkanes to alkenes and/or to dienes, it should be noted that the invention is also suitable for dehydrogenation of other hydrocarbons. Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. Furthermore, individual features mentioned in different claims may possibly be advantageously combined, and the mentioning of these features in different claims does not exclude that a combination of features is not possible and advantageous.

1. A reactor system for dehydrogenation of ethylbenzene to styrene in a given temperature range T upon bringing a reactant stream comprising ethylbenzene into contact with a catalytic mixture, said reactor system comprising:
a reactor unit arranged to accommodate said catalytic mixture, said catalytic mixture comprising catalyst particles in intimate contact with a ferromagnetic material, where said catalyst particles are arranged to catalyze the dehydrogenation of ethylbenzene to styrene,
an induction coil arranged to be powered by a power source supplying alternating current and being positioned so as to generate an alternating magnetic field within the reactor unit upon energization by the power source, whereby the catalytic mixture is heated to a temperature within said temperature range T by means of said alternating magnetic field.

2. A reactor system according to claim 1, wherein the given temperature range T is the range between about 450° C. and about 650° C.

3. A reactor system according to claim 1, wherein the Curie temperature of the ferromagnetic material equals an operating temperature at substantially the upper limit of the given temperature range T of the dehydrogenation reaction.

4. A reactor system according to claim 1, wherein the Curie temperature of the ferromagnetic material is above 450° C.

5. A reactor system according to claim 1, wherein the induction coil is placed within the reactor unit or around the reactor unit.

6. A reactor system according to claim 1, wherein said catalyst particles are supported on said ferromagnetic material.

7. A reactor system according to claim 6, wherein said ferromagnetic material comprises one or more ferromagnetic macroscopic supports susceptible for induction heating, where said one or more ferromagnetic macroscopic supports are ferromagnetic at temperatures up to an upper limit of the given temperature range T, where said one or more ferromagnetic macroscopic supports is/are coated with an oxide and where the oxide is impregnated with said catalyst particles.

8. A reactor system according to claim 1, wherein catalyst particles and ferromagnetic particles are mixed and treated to provide bodies of catalytic mixture.

9. A reactor system according to claim 1, wherein said catalytic mixture comprises bodies of catalyst particles mixed with bodies of ferromagnetic material, wherein the smallest outside dimension of the bodies are in the order of about 1-2 mm or larger.

10. A reactor system according to claim 1, wherein the catalytic mixture has a predetermined ratio between said catalyst particles and said ferromagnetic material.

11. A reactor system according to claim 10, wherein the predetermined ratio between said catalyst particles and said ferromagnetic material is a predetermined graded ratio varying along a flow direction of said reactor.

12. A catalytic mixture arranged for catalyzing dehydrogenation of ethylbenzene in a reactor in a given temperature range T upon bringing a reactant stream comprising ethylbenzene into contact with said catalytic mixture, said catalytic mixture comprising catalyst particles in intimate contact with a ferromagnetic material, where said catalyst particles are arranged to catalyze the dehydrogenation of ethylbenzene to styrene.

13. A catalytic mixture according to claim 12, wherein the Curie temperature of the ferromagnetic material substantially equals an operating temperature at substantially the upper limit of the given temperature range T of the dehydrogenation reaction.

14. A catalytic mixture according to claim 12, wherein the ferromagnetic material is an alloy comprising iron, an alloy comprising iron and chromium, an alloy comprising iron, chromium and aluminum, an alloy comprising iron and cobalt, an alloy comprising iron, aluminum, nickel and cobalt, or magnetite.

15. A catalytic mixture according to claim 12, wherein the catalyst particles comprise a carrier impregnated with an iron oxide and/or a potassium oxide, optionally promoted by a cerium oxide and/or a molybdenum oxide.

16. A catalytic mixture according to claim 12, wherein said catalyst particles are supported on said ferromagnetic material.

17. A catalytic mixture according to claim 16, wherein said ferromagnetic material comprises one or more ferromagnetic macroscopic supports susceptible for induction heating, where said one or more ferromagnetic macroscopic supports are ferromagnetic at temperatures up to an upper limit of the given temperature range T, where said one or more ferromagnetic macroscopic supports is/are coated with an oxide and where the oxide is impregnated with catalyst particles.

18. A catalytic mixture according to claim 12, wherein catalyst particles and ferromagnetic particles are mixed and treated to provide bodies of catalytic mixture.

19. A catalytic mixture according to claim 12, wherein said catalytic mixture comprises bodies of catalyst particles mixed with bodies of ferromagnetic material.

20. A catalytic mixture according to claim 12, wherein the catalytic mixture has a predetermined ratio between said catalyst particles and said ferromagnetic material.

21. A catalytic mixture according to claim 20, wherein the predetermined ratio between said catalyst particles and said ferromagnetic material is a predetermined graded ratio varying along a flow direction of said reactor.

22. A method for dehydrogenating of ethylbenzene in a given temperature range T in a reactor system, said reactor system comprising a reactor unit arranged to accommodate a catalytic mixture, said catalytic mixture comprising catalyst particles in intimate contact with a ferromagnetic material, where said catalyst particles are arranged to catalyze the dehydrogenation of ethylbenzene to styrene, and an induction coil arranged to be powered by a power source supplying alternating current and positioned so as to generate an alternating magnetic field within the reactor unit upon energization by the power source, whereby the catalytic mixture is heated to a temperature within the given temperature range T by means of said alternating magnetic field, said method comprising the steps of:
Generating an alternating magnetic field within the reactor unit upon energization by a power source supplying alternating current, said alternating magnetic field passing through the reactor unit, thereby heating catalytic mixture by induction of a magnetic flux in the material;
bringing a reactant stream comprising ethylbenzene into contact with said catalyst particles;
heating said reactant stream within said reactor by the generated alternating magnetic field; and
letting the reactant stream react in order to provide a styrene product stream to be outlet from the reactor.

23. A method according to claim 22, wherein the temperature range T is the range from between about 450° C. and about 650° C. or a subrange thereof.

24. A method according to claim 22, wherein the reactant stream is preheated in a heat exchanger prior to step (ii).

25. A method according to claim 22, wherein the reactant stream comprises steam and wherein the mass ratio between steam and ethylbenzene in the reactant stream is a ratio of steam/ethylbenzene between 0.5 and 1.5 (wt/wt).
